(19) United States
(12) Patent Application Publication
(10) Pub. No.: US 2025/0290842 A1
(43) Pub. Date: Sep. 18, 2025

(54) SYSTEMS AND METHODS FOR CHARACTERIZING UNCONVENTIONAL RESERVOIRS INCLUDING FRACTURED SOURCE ROCK

(71) Applicant: ARAMCO SERVICES COMPANY, Houston, TX (US)

(72) Inventors: Hui-Hai Liu, Katy, TX (US); Jilin Zhang, Houston, TX (US); Rabah Mesdour, Dhahran (SA); Mohammad Abdullah AlQarni, Dhahran (SA)

(73) Assignees: ARAMCO SERVICES COMPANY, Houston, TX (US); SAUDI ARABIAN OIL COMPANY, Dhahran (SA)

(21) Appl. No.: 18/602,309

(22) Filed: Mar. 12, 2024

(51) Int. Cl. G01N 15/08; G01N 33/24

(52) U.S. Cl. CPC G01N 15/0826; G01N 33/24

(57) ABSTRACT

Systems, methods, and apparatus for characterizing reservoirs are discussed. As an example, a method for characterizing a reservoir is discussed that includes determining a kᵢ parameter, a α_{E} parameter, and a α_{P} parameter based at least in part on the at least four pairs of effective stress and permeability; and predicting a hydrocarbon production rate from reservoir represented by the sample rock based at least in part on the kᵢ parameter, the α_{E} parameter, and the α_{P} parameter.

### TECHNICAL FIELD

This application relates to fracture permeability. More particularly, the application relates to modelling for stress sensitive fracture permeability.

### BACKGROUND

One important parameter impacting estimated ultimate recovery (EUR) of hydrocarbon from unconventional reservoirs is stimulated reservoir volume. The volume is generated by hydraulic fracturing and obtaining a complex network of hydraulic fractures and sometimes natural fractures as well. The large interfacial area between the fractures and rock matrix and relatively high fracture permeability makes the production economically feasible from unconventional reservoirs, although the rock matrix permeability is extremely low.

It is well known that fracture permeability is stress sensitive, simply because fracture is less stiff than unfractured rock mass. Thus, production rate is largely controlled by the evolution of fracture permeability within stimulated reservoir volume. During production, the stimulated reservoir volume pore pressure generally decreases, while temporal pressure increase exists as well because well bottom hole pressure can fluctuate owing to operation considerations. The pore pressure change can result in complex behavior of the stress-dependent permeability associated with a combination of rock elastic and plastic deformations, while previous models of fracture permeability as a function stress have been mainly for elastic deformation.

Hence, there exists a need in the art for advanced systems and methods for characterizing fracture permeability as a function of stress and/or plastic deformations.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

Some embodiments provide methods for characterizing unconventional reservoirs. In some cases the methods may include: using a first pump and a first pressure transducer on a first side of a core holder holding a sample rock and a second pump and a second pressure transducer on a second side of the core holder to obtain at least four pairs of effective stress and permeability values for the sample rock, where the sample rock comprises fractures; determining a kᵢ parameter, a α_{E} parameter, and a α_{P} parameter based at least in part on the at least four pairs of effective stress and permeability; and predicting a hydrocarbon production rate from reservoir represented by the sample rock based at least in part on the kᵢ parameter, the α_{E} parameter, and the α_{P} parameter. The kᵢ is an initial permeability of the sample rock before any elastic or plastic deformation of the sample rock, the α_{P} is stress sensitivity parameter for permeability associated with plastic deformation of the sample rock, and the αE is a stress sensitivity parameter for permeability associated with elastic deformation of the sample rock.

Various embodiments provide systems for characterizing unconventional reservoirs. In some cases the systems may include: a core holder configured to hold a sample rock, where the sample rock comprises fractures; a first pump; a first pressure transducer, where the first pump and the first pressure transducer are located on an inlet side of the core holder; a second pump; a second pressure transducer, where the second pump and the second pressure transducer are located on an outlet side of the core holder; a third pump; a third pressure transducer, where the third pump is configured to control an effective stress on the sample rock, and where the third pressure transducer is configured to sense the pressure generated by the third pump on the sample rock; and a reservoir characterization device. The reservoir characterization device may be configured to: receive first pressure measurements from the first pressure transducer, second pressure measurements from the second pressure transducer, and third pressure measurements from the third pressure transducer; generate at least four pairs of effective stress and permeability based upon a combination of the first pressure measurements, the second pressure measurements, and the third pressure measurements; determine a kᵢ parameter, a α_{E} parameter, and a α_{P} parameter based at least in part on the at least four pairs of effective stress and permeability; where ki is an initial permeability of the sample rock before any elastic or plastic deformation of the sample rock, α_{P} is stress sensitivity parameter for permeability associated with plastic deformation of the sample rock, and αE is a stress sensitivity parameter for permeability associated with elastic deformation of the sample rock; and predict a hydrocarbon production rate from reservoir represented by the sample rock based at least in part on the kᵢ parameter, the α_{E} parameter, and the α_{P} parameter.

Some embodiments provide computer readable media. The computer readable media may have stored therein instructions which, when executed by one or more processors perform the following method: receiving first pressure measurements from a first pressure transducer measuring pressure at an inlet side of a core holder holding a sample rock; receiving second pressure measurements from a second pressure transducer measuring pressure at an outlet side of the core holder; receiving third pressure measurements from a third pressure transducer measuring an effective stress on the sample rock; generating at least four pairs of effective stress and permeability based upon a combination of the first pressure measurements, the second pressure measurements, and the third pressure measurements; determining a kᵢ parameter, an α_{E} parameter, and an α_{P} parameter based at least in part on the at least four pairs of effective stress and permeability; where the kᵢ parameter is an initial permeability of the sample rock before any elastic or plastic deformation of the sample rock, the α_{P} parameter is stress sensitivity parameter for permeability associated with plastic deformation of the sample rock, and the α_{E} parameter is a stress sensitivity parameter for permeability associated with elastic deformation of the sample rock; and predicting a hydrocarbon production rate from reservoir represented by the sample rock based at least in part on the kᵢ parameter, the α_{E} parameter, and the α_{P} parameter.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows an unconventional reservoir characterization system employing a steady-state flow method in accordance with some embodiments.

FIG. 2 is a graphic depicting a pattern of loading and unloading of effective stress for a fresh fracture.

FIG. 3 is a graphic depicting a pattern of loading and unloading of effective stress for pre-stressed fractures.

FIG. 4 is a graphic depicting a pattern of loading of effective stress in accordance with some embodiments.

FIG. 5 shows another unconventional reservoir characterization system employing a pressure pulse decay method in accordance with various embodiments.

FIG. 6 shows a computer system in accordance with one or more embodiments.

FIG. 7 is a flow diagram showing a method in accordance with some embodiments for predicting hydrocarbon production from an unconventional reservoir.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

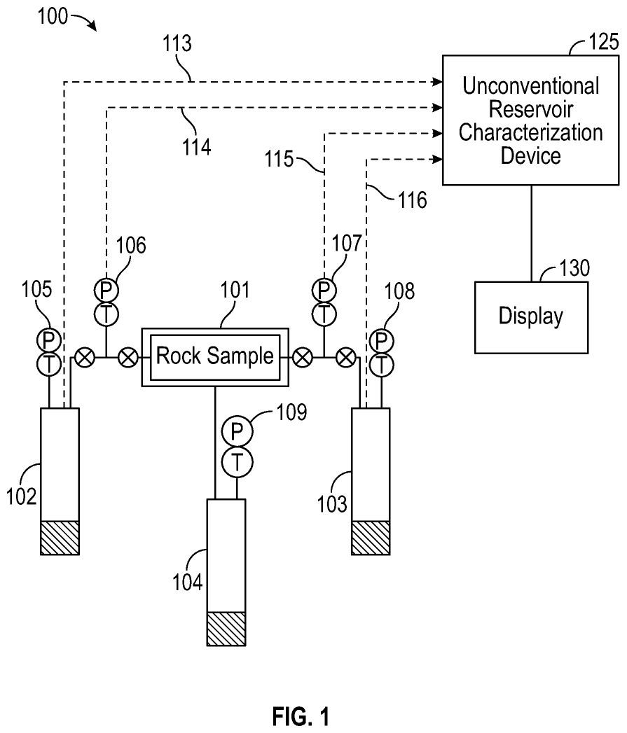

In the following description of FIGS. 1-7, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

It is to be understood that the singular forms “a,” “an,” and “the” include plural referents unless the context clearly dictates otherwise. Thus, for example, reference to “a circuit breaker” includes reference to one or more of such circuit breakers.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

Turning to FIG. 1, an unconventional reservoir characterization system 100 is shown that uses a steady-state flow method in accordance with some embodiments. As shown, unconventional reservoir characterization system 100 includes a core holder 101. Core holder 101 is configured to hold a rock sample. This rock sample may be, but is not limited to, a rock sample from an unconventional reservoir under investigation. Such a rock sample may include fractures. The fractures in the rock sample may be caused, for example, by a fracturing process used in developing the unconventional reservoir.

In addition, unconventional reservoir characterization system 100 includes: an upstream pump 102, a downstream pump 103, a confining pump 104, a first transducer pair 105 including a pressure transducer (P) and a temperature transducer (T), a second transducer pair 106 including a pressure transducer (P) and a temperature transducer (T), a third transducer pair 107 including a pressure transducer (P) and a temperature transducer (T), a fourth transducer pair 108 including a pressure transducer (P) and a temperature transducer (T), and a fifth transducer pair 109 including a pressure transducer (P) and a temperature transducer (T).

Pressure and temperature at upstream pump 102 are measured by first transducer pair 105. Pressure and temperature at the inlet of core holder 101 are measured by second transducer pair 106. Pressure and temperature at the outlet of core holder 101 are measured by third transducer pair 107. Pressure and temperature at downstream pump 103 are measured by fourth transducer pair 108. Pressure and temperature at confining pump 104 are measured by fifth transducer pair 109. In some embodiments, all of the pressure transducers (P) may be the same type of pressure transducers, and all of the temperature transducers (T) may be the same type of temperature transducers. Based upon the disclosure provided herein, one of ordinary skill in the art will recognize a variety of pressure and temperature transducers that may be used in relation to different embodiments.

Confining pump 104 is fluidically coupled to core holder 101. When engaged, confining pump 104 causes a confining pressure to build on a confining fluid within core holder 101. This confining fluid within core holder 101 is separated from a rock sample in core holder 101 by seals.

An inlet to a rock sample within core holder 101 is fluidically coupled to upstream pump 102, and an outlet from the rock sample within core holder 101 is fluidically coupled to downstream pump 103. When engaged, upstream pump 102 and downstream pump 103 work together to maintain constant upstream and downstream fluid pressures to/from core holder 101. Upstream pump 102 includes an incorporated flow sensor configured to measure a flow of fluid out of upstream pump 102. Similarly, downstream pump 103 includes an incorporated flow sensor configured to measure a flow of fluid into downstream pump 103.

Upstream pump 102, downstream pump 103, and confining pump 104 may be any pumps known in the art that are capable of generating sufficient flow and pressure to achieve a steady-state flow through the rock sample in core holder 101. Based upon the disclosure provided herein, one of ordinary skill in the art will recognize a variety of pumps that may be used in relation to different embodiments.

In operation, each of upstream pump 102, downstream pump 103, and confining pump 104 are engaged to create a pressurized system. Unconventional reservoir characterization system 100 is placed in an oven creating an environment where the ambient temperature is both controlled and substantially constant to ensure isothermal test conditions such that the fluid flow can be described as a function of a pressure gradient across the rock sample. When a first mass flow rate from upstream pump 102 into an inlet of the rock sample within core holder 101 and a second mass flow rate into downstream pump 103 from the outlet of the rock sample within core holder 101 are substantially equal, a steady-state flow has been reached.

A unconventional reservoir characterization device 125 monitors a volumetric flow data 113 measured by and transmitted from a flow sensor in upstream pump 102 and a volumetric flow data 116 measured by and transmitted from a flow sensor in downstream pump 103. Unconventional reservoir characterization device 125 multiplies the received volumetric flow rates by local fluid densities that depend on pressure and temperature data 114 transmitted in real-time from second transducer pair 106 and pressure and temperature data 114 transmitted in real-time from third transducer pair 107. Unconventional reservoir characterization device 125 uses the resulting mass flow rate calculated from volumetric flow data 113 and the resulting mass flow rate calculated from volumetric flow data 116 to determine whether unconventional reservoir characterization system 100 is operating in a steady-state flow (i.e., determine whether the respective mass flow rates are approximately equal).

In some embodiments, volumetric flow data 113, pressure and temperature data 114, pressure and temperature data 115, and volumetric flow data 116 are transmitted via wireless communication links. In other embodiments, volumetric flow data 113, pressure and temperature data 114, pressure and temperature data 115, and volumetric flow data 116 are transmitted via wired communication links. Based upon the disclosure provided herein, one of ordinary skill in the art will recognize a variety of wired and/or wireless communication links and corresponding transmission protocols that may be used in relation to different embodiments.

Once operation in the steady-state flow is achieved, unconventional reservoir characterization device 125 updates a permeability value (k) in real-time as updated temperature and pressure data 114, 115 is received using Darcy's law represented by the following equation:

$$Q = ( k ρΔ p / μ ) ( A / L ) ,$$

where A is the cross-sectional area of the rock sample, L is the length of the rock sample, Q is the mass flow rate, ρ is the viscosity of the fluid flowing from upstream pump 102 to downstream pump 103, and Δp is the pressure differential between the pressure component of pressure and temperature data 114 and the pressure of component of pressure and temperature data 115. Unconventional reservoir characterization device 125 provides the calculated permeability value to a display 130. Display 130 may be any device known in the art for displaying information.

In addition, unconventional reservoir characterization device 125 determines model parameters using the calculated permeability value. The model parameters are used to model fracture permeability in an unconventional reservoir as a function of stress that considers the effects of both elastic and plastic deformations. In some embodiments, modeling fracture permeability in the unconventional reservoir is based upon an effective stress (σₑ) defined as: σₑ=σ_{c}−δP, where σ_{c} is confining stress (or total stress), δ is Biot's coefficient that is a constant between zero and one, and P is the pore pressure. Confining stress (σ_{c}) is controlled by confining pump 104 pressurizing a confining fluid in core holder 101.

For elastic deformation, permeability (k) of the rock sample can be expressed in terms of the effective stress (σₑ) as follows: k=k₀exp(−α_{E}σₑ), where k₀ is the permeability of the rock sample when the effective stress is zero, and permeability under zero effective stress, and α_{E} is a stress sensitivity parameter for permeability that is a positive constant.

Where plastic deformation exists, permeability (k) of the rock sample can be expressed in terms of both stress and stress history. Where both plastic and elastic deformation are present, k₀ for the rock sample cannot be considered a constant, but rather is a function of a maximum stress (σₘ) that the same rock has experienced as follows: k₀=kᵢexp(−α_{P}σₑ), where kᵢ is the initial rock permeability before any elastic and plastic deformation, and α_{P} is the stress sensitivity parameter for permeability associated with plastic deformation. Thus, the permeability (k) of the sample rock exhibiting both plastic and elastic deformation can be expressed as: k=kᵢ*exp(−α_{P}σₘ)*exp(−α_{E}σₑ).

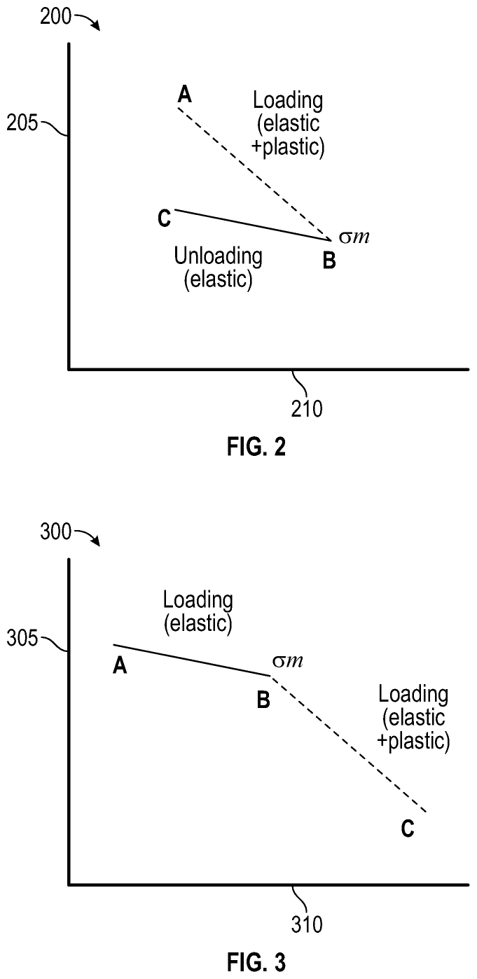

Turning to FIG. 2, a graphic 200 depicts a pattern of loading and unloading of effective stress for a fresh fracture. As shown, natural log (ln) of permeability is shown on a vertical axis 205 and the corresponding effective stress is shown on a horizontal axis 210. For fresh fractures, σₘ=σₑ for a loading test depicted in graphic 200 that involves both elastic and plastic deformation. Using the preceding equation describing permeability as a function of both σₘ and σₑ, the permeability from a stress point A to a stress point B is expressed as: ln(k)=ln(kᵢ)−(α_{P}+α_{E})*σₑ Thus, the slope of the loading curve is −(α_{P}+α_{E}).

At stress point B, unloading starts to occur. Since the largest effective stress that the sample rock has experienced is the effective stress at stress point B (or σₘ), during the unloading σₘ is a constant and the deformation is elastic. As such, the aforementioned expression of permeability reduces to: ln(k)=Constant−α_{E}*σₑ. The slope of the unloading curve from stress point B to a stress point C is thus −α_{E}.

Turning to FIG. 3, a graphic 300 depicts a pattern of loading and unloading of effective stress for a pre-stressed fracture. As shown, natural log (ln) of permeability is shown on a vertical axis 305 and the corresponding effective stress is shown on a horizontal axis 310. In the graphic, the rock sample is pre-stressed to an effective stress equal to that at stress point B of FIG. 2 (stress points A, B, and C are also shown in FIG. 3). Thus, when loading begins from stress point A to stress point B, all the effective stresses are smaller than the maximum stress (or σₘ) that the sample rock has experienced. The deformation is elastic and permeability relation is described as: ln(k)=Constant−α_{E}*σₑ. The slope of the curve from stress point A to stress point B is −α_{E}. During further loading from stress point B to stress point C, the maximum stress (or σₘ) is not a constant any longer, but is the same as σₑ. Thus, in graphic 300 the unloading from stress point B to stress point C is subject to both elastic and plastic deformation and has a slope of −(α_{P}+α_{E}) as explained above.

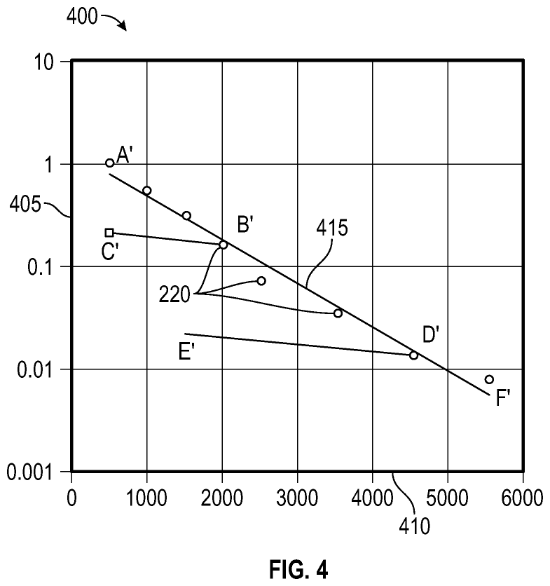

Turning to FIG. 4, a graphic 400 depicts a pattern of loading/unloading of effective stress in accordance with some embodiments. As shown, normalized permeability is shown on a vertical axis 405 and the corresponding effective stress is shown on a horizontal axis 410. A line 415 is mapped to a number of combinations of pairs of permeability and effective stress are shown as dots (e.g., dots 220). Loading of the sample rock begins from a stress point A′ and continues to a stress point B′ where both elastic and plastic deformation are present. Unloading occurs from stress point B′ to a stress point C′ subject to only elastic deformation. After reloading from stress point C′ to stress point B′, loading continues from stress point B′ to a stress point D′ where both elastic and plastic deformation are present. Unloading is repeated from stress point D′ to a stress point E′ subject to only elastic deformation. After reloading from stress point E′ to stress point D′, loading continues from stress point D′ to a stress point F′ where both elastic and plastic deformation are present.

Returning to FIG. 1, The loading, unloading, and reloading processes described in relation to FIG. 4 may be applied to a sample rock in core holder 101 through control of confining pump 104 by unconventional reservoir characterization device 125. The loading, unloading, and reloading processes are repeated to obtain a set of permeability measurements for the sample rock at different effective stresses. The obtained permeability measurements are stored in a memory of unconventional reservoir characterization device 125. In some embodiments, the process begins with an initial effective stress and continues to an effective stress of at least two times the initial effective stress. In some embodiments, the initial effective stress is five hundred pounds per square inch (500 psi). Based upon the disclosure provided herein, one of ordinary skill in the art will recognize a variety of ranges of effective stress that may be used in relation to different embodiments.

Unconventional reservoir characterization device 125 fits the obtained set of permeability measurements for the sample rock at different effective stresses to expected elastic and plastic deformations (e.g., moving from stress point A to stress point B in FIG. 2) to determine kᵢ and (α_{P}+α_{E}). Said another way, unconventional reservoir characterization device 125 determines kᵢ and (α_{P}+α_{E}), by fitting the obtained set of permeability measurements to the equation ln(k)=ln(kᵢ)−(α_{P}+α_{E})*σₑ.

Further, unconventional reservoir characterization device 125 fits the obtained set of permeability measurements for the sample rock at different effective stresses to expected elastic deformations (e.g., moving from stress point B to stress point C in FIG. 2 and moving from stress point A to stress point B in FIG. 3) to determine α_{E}. Said another way, unconventional reservoir characterization device 125 determines α_{E}, by fitting the obtained set of permeability measurements to the equation ln(k)=Constant−α_{E}*σₑ. The parameter α_{P} may be determined by subtracting the determined α_{E} from the determined (α_{P}+α_{E}). The parameters (i.e., kᵢ, α_{P}, and α_{E}) are stored to the memory of unconventional reservoir characterization device 125.

As the production rate in an unconventional reservoir is largely controlled by the evolution of fracture permeability within a stimulated reservoir volume, unconventional reservoir characterization device 125 may use the parameters (i.e., kᵢ, α_{P}, and α_{E}) to predict hydrocarbon production from an unconventional reservoir represented by the sample rock. Unconventional reservoir characterization device 125 displays the predicted hydrocarbon production via display 130.

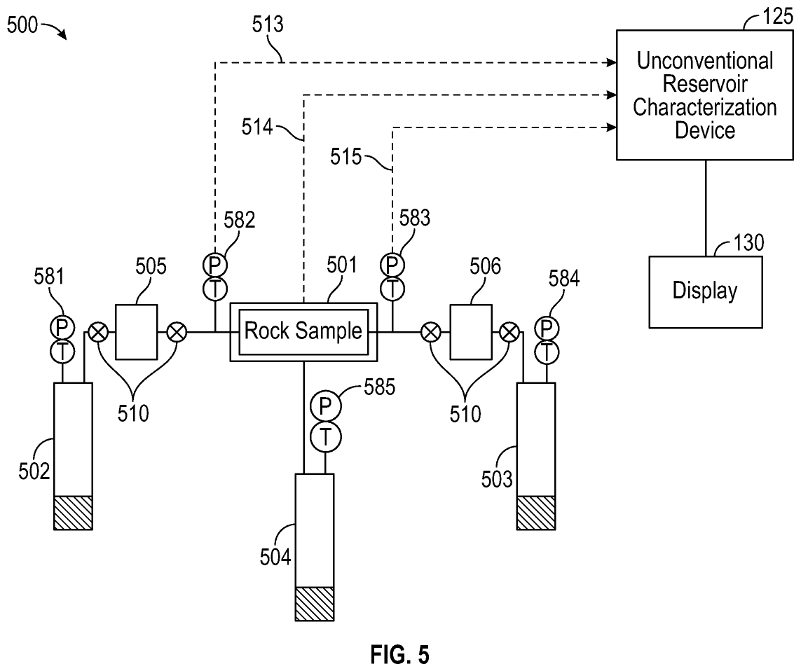

Turning to FIG. 5, another unconventional reservoir characterization system 500 employing a pressure pulse decay method in accordance with various embodiments. As shown, unconventional reservoir characterization system 500 includes a core holder 501 configured to hold a rock sample. This rock sample may be, but is not limited to, a rock sample from an unconventional reservoir under investigation. Such a rock sample may include fractures. The fractures in the rock sample may be caused, for example, by a fracturing process used in developing the unconventional reservoir.

In addition, unconventional reservoir characterization system 500 includes: an upstream pump 502, a downstream pump 503, a confining pump 504, a first transducer pair 581 including a pressure transducer (P) and a temperature transducer (T), a second transducer pair 582 including a pressure transducer (P) and a temperature transducer (T), a third transducer pair 583 including a pressure transducer (P) and a temperature transducer (T), a fourth transducer pair 584 including a pressure transducer (P) and a temperature transducer (T), and a fifth transducer pair 585 including a pressure transducer (P) and a temperature transducer (T). Unconventional reservoir characterization system 500 also includes: an upstream reservoir 505 connected to an inlet of core holder 501, and a downstream reservoir 506 connected to an outlet of core holder 501. Valves 510 control the flow of fluids in unconventional reservoir characterization system 500.

Pressure and temperature at upstream pump 502 are measured by first transducer pair 581. Pressure and temperature at the inlet of core holder 101 are measured by second transducer pair 582. Pressure and temperature at the outlet of core holder 101 are measured by third transducer pair 583. Pressure and temperature at downstream pump 503 are measured by fourth transducer pair 584. Pressure and temperature at confining pump 504 are measured by fifth transducer pair 585. In some embodiments, all of the pressure transducers (P) may be the same type of pressure transducers, and all of the temperature transducers (T) may be the same type of temperature transducers. Based upon the disclosure provided herein, one of ordinary skill in the art will recognize a variety of pressure and temperature transducers that may be used in relation to different embodiments.

Confining pump 504 is fluidically coupled to core holder 501. When engaged, confining pump 504 causes a confining pressure to build on a confining fluid within core holder 501. This confining fluid within core holder 501 is separated from a rock sample in core holder 501 by seals.

An inlet to the rock sample within core holder 501 is fluidically coupled to upstream reservoir 505, and upstream reservoir 505 is fluidically coupled to upstream pump 502. An outlet from the rock sample within core holder 501 is fluidically coupled to downstream reservoir 506, and downstream reservoir 506 is fluidically coupled to downstream pump 503. Control of fluid movement is governed by valves 510. Upstream pump 502 includes an incorporated flow sensor configured to measure a flow of fluid out of upstream pump 502. Similarly, downstream pump 503 includes an incorporated flow sensor configured to measure a flow of fluid into downstream pump 503.

Upstream pump 502, downstream pump 503, and confining pump 504 may be any pumps known in the art that are capable of generating sufficient flow and pressure to achieve a steady-state flow through the rock sample in core holder 501. Based upon the disclosure provided herein, one of ordinary skill in the art will recognize a variety of pumps that may be used in relation to different embodiments.

In operation, unconventional reservoir characterization device 125 receives in real-time: pressure and temperature data from second transducer pair 582 via a communication link 513, pressure and temperature data from third transducer pair 583 via a communication link 515, and pressure and temperature data from fifth transducer pair 585 via a communication link 515. Valves 510 are opened and both upstream pump 502 and downstream pump 503 are engaged such that a working fluid used for measuring permeability of the rock sample is injected through core holder 501 via the inlet and the outlet. Unconventional reservoir characterization device 125 controls upstream pump 502 and downstream pump 503 based upon pressure data received from second transducer pair 582 and third second transducer pair 583 to equilibrate a pore pressure within the rock sample at a desired initial pressure. Depending on the permeability value for the rock sample, this equilibrium process can take several hours or days for rock samples that are tight.

Once equilibrium is achieved at the desired pressure, upstream reservoir 505 is disconnected from the rock sample by closing the valve 510 between upstream reservoir 505 and the inlet of core holder 501. A gas pressure pulse is created in upstream reservoir 505 by injecting a certain amount of the working fluid. A small pressure pulse is generally used such that in upstream reservoir 505 a pressure increase is a few percent of the initial gas pressure. The pressure pulse decay method relies on the analytical solutions to linear or linearized gas flow equations that require small disturbances to the test system such that fluid density and viscosity can be approximated as constants within the rock sample.

After the pressure in upstream reservoir 505 reaches a desired value, the valve 510 between upstream reservoir 505 and the inlet of core holder 501 is re-opened allowing the gas from upstream reservoir 505 to flow through the rock sample in core holder 501 to downstream reservoir 506. Pressure data from second transducer pair 582 and third second transducer pair 583 are monitored by unconventional reservoir characterization device 125 as a function of time to yield an evolution of gas pressures as a function of time. Other than unconventional reservoir characterization device 125, unconventional reservoir characterization system 500 is maintained under isothermal conditions during the pressure pulse decay testing process.

Unconventional reservoir characterization device 125 determines permeability of the sample rock by fitting the pressure data from second transducer pair 582 and third second transducer pair 583 with a relevant analytical solution to fluid flow within the test system, with permeability being a fitting parameter. Since fluid and rock properties could be approximated as constants, the governing equation for working fluid flow is linear and can be solved analytically using the following equation:

ln(pᵤ−p_{d})=−fkt+C, where pᵤ is the pressure data from second transducer pair 582 and p_{d} is the pressure data from third transducer pair 583. f is a function of the geometry of the sample rock (i.e., length and cross-sectional area), rock porosity, and properties of the working fluid (i.e., density, viscosity, and compressibility). Fitting the pressure data from second transducer pair 582 and third transducer pair 583 with the aforementioned equation yields the permeability value of the sample rock the given pore pressure and confining stress (or pressure).

Unconventional reservoir characterization device 125 determines if the observed permeability-stress relation from the loading process follows curves in FIG. 3 or if it follows the curve from stress point A to stress point B in FIG. 2. Where the permeability more closely follows the curve from stress point A to stress point B in FIG. 2, additional loading is applied by confining pump 504 and the process of determining permeability of the sample rock is repeated.

Alternatively, where the permeability more closely follows the curve in FIG. 3, unconventional reservoir characterization device 125 stores the permeability values in its memory. Unconventional reservoir characterization device 125 fits the obtained set of permeability measurements for the sample rock at different effective stresses to expected elastic and plastic deformations (e.g., moving from stress point A to stress point B in FIG. 2) to determine kᵢ and (α_{P}+α_{E}). Said another way, unconventional reservoir characterization device 125 determines kᵢ and (αₚ+α_{E}), by fitting the obtained set of permeability measurements to the equation ln(k)=ln(kᵢ)−(α_{P}+α_{E})*σₑ.

Further, unconventional reservoir characterization device 125 fits the obtained set of permeability measurements for the sample rock at different effective stresses to expected elastic deformations (e.g., moving from stress point B to stress point C in FIG. 2 and moving from stress point A to stress point B in FIG. 3) to determine α_{E}. Said another way, unconventional reservoir characterization device 125 determines α_{E}, by fitting the obtained set of permeability measurements to the equation ln(k)=Constant−α_{E}*σₑ. The parameter α_{P} may be determined by subtracting the determined α_{E} from the determined (α_{P}+α_{E}). The parameters (i.e., kᵢ, α_{P}, and α_{E}) are stored to the memory of unconventional reservoir characterization device 125.

As the production rate in an unconventional reservoir is largely controlled by the evolution of fracture permeability within a stimulated reservoir volume, unconventional reservoir characterization device 125 may use the parameters (i.e., kᵢ, α_{P}, and α_{E}) to predict hydrocarbon production from an unconventional reservoir represented by the sample rock. Unconventional reservoir characterization device 125 displays the predicted hydrocarbon production via display 130.

In some embodiments, the production rate may be included as part of a reservoir simulation. Such reservoir simulation plays an important role in planning and managing a hydrocarbon reservoir. Simulation typically involves representing the properties (e.g., rock porosity and permeability, and pore fluid pressure) of the reservoir on a grid of points. In an initial stage, termed “history matching”, past (“historic”) production, i.e., flow rates and downhole pressures, may be simulated and reservoir properties adjusted, iteratively, until simulated values match the historic measured values of flow rates and downhole pressures. In a second predictive phase, the behavior of the reservoir, including pore fluid pressure and phase changes of the fluid within the reservoir and production rates, may be simulated for various downhole pressure scenarios. Simulations may include cases where additional wellbore are drilled and/or fluid injection schedules are implemented. These predictions help inform the future operation of the reservoir.

A reservoir simulation model may be used by a reservoir simulator to perform reservoir simulation. The reservoir simulator may include computer system, such as the computer system described later, and software with functionality for simulating percolation or the flow of reservoir fluid through the reservoir in response to natural or anthropogenic pressure gradients.

For example, the reservoir simulator may solve a set of mathematical governing equations that represent the physical laws that govern fluid flow in porous, permeable media. For example, the flow of a single-phase slightly compressible oil with a constant viscosity and compressibility is captured by Darcy's law, the continuity condition and the equation of state and may be written as:

$$∇ \cdot ( \frac{k}{μ} ∇ p ( x , t ) ) = φ {c}_{t} \frac{\partial p ( x , t )}{\partial t}$$

where p represents fluid in the reservoir, x is a vector representing spatial position and t represents time. φ, μ, cₜ, and k represent the physical and petrophysical properties of porosity, fluid viscosity, total combined rock and fluid compressibility, and permeability, respectively. ∇² represents the spatial Laplacian operator.

Additional, and more complicated equations, such as the Peng-Robinson EoS, may be required when more than one fluid, or more than one phase, e.g., liquid and gas, are present in the reservoir. For example, the formation of “condensates”, i.e., oil condensing from a vapor state, gas, or outgassing of natural gas previously dissolved within a hydrocarbon liquid may be simulated.

Further, when the physical and petrophysical properties of the rocks and fluids vary as a function of position the governing equations may not be solved analytically and must instead be discretized into a grid of cells or blocks. The governing equations may then be solved by one of a variety of numerical methods well known to one of ordinary skill in the art, such as, without limitation, explicit or implicit finite-difference methods, explicit or implicit finite element methods, or discrete Galerkin methods.

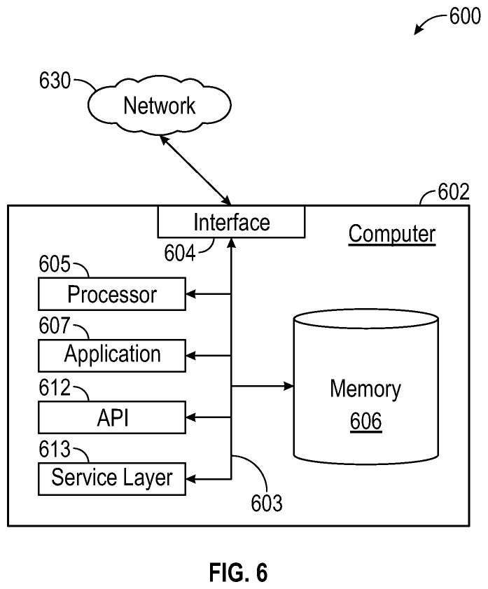

Turning to FIG. 6, a block diagram of a computer system 602 is provided. Computer system 602 may be used to provide computational functionalities associated with described algorithms, methods, functions, processes, flows, and procedures as described in this disclosure, according to one or more embodiments. For example, the computer system 602, and the processor of the computer system, may be used to perform one or more processes discussed above in relation to FIGS. 1 and 5.

Computer system 602 is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer system 602 may include a computer that includes an input device, such as a keypad, keyboard, touch screen, or other device that can accept user information, and an output device that conveys information associated with the operation of the computer system 602, including digital data, visual, or audio information (or a combination of information), or a graphical user interface.

Computer system 602 can serve in a role as a client, network component, a server, a database or other persistency, or any other component (or a combination of roles) of a computer system for performing the subject matter described in the instant disclosure. Computer system 602 is communicably coupled with a network 630. In some implementations, one or more components of computer system 602 may be configured to operate within environments, including cloud-computing-based, local, global, or other environment (or a combination of environments).

At a high level, computer system 602 is an electronic computing device operable to receive, transmit, process, store, or manage data and information associated with the described subject matter. According to some implementations, the computer system 602 may also include or be communicably coupled with an application server, e-mail server, web server, caching server, streaming data server, business intelligence (BI) server, or other server (or a combination of servers).

Computer system 602 can receive requests over network 630 from a client application (for example, executing on another computer system 602) and responding to the received requests by processing the said requests in an appropriate software application. In addition, requests may also be sent to computer system 602 from internal users (for example, from a command console or by other appropriate access method), external or third-parties, other automated applications, as well as any other appropriate entities, individuals, systems, or computers.

Each of the components of computer system 602 can communicate using a system bus 603. In some implementations, any or all of the components of computer system 602, both hardware or software (or a combination of hardware and software), may interface with each other or the interface 604 (or a combination of both) over the system bus 603 using an application programming interface (API) 612 or a service layer 613 (or a combination of the API 612 and service layer 613. The API 612 may include specifications for routines, data structures, and object classes. The API 612 may be either computer-language independent or dependent and refer to a complete interface, a single function, or even a set of APIs. The service layer 613 provides software services to the computer system 602 or other components (whether or not illustrated) that are communicably coupled to the computer system 602. The functionality of the computer system 602 may be accessible for all service consumers using this service layer. Software services, such as those provided by the service layer 613, provide reusable, defined business functionalities through a defined interface. For example, the interface may be software written in JAVA, C++, or other suitable language providing data in extensible markup language (XML) format or another suitable format. While illustrated as an integrated component of the computer system 602, alternative implementations may illustrate the API 612 or the service layer 613 as stand-alone components in relation to other components of computer system 602 or other components (whether or not illustrated) that are communicably coupled to computer system 602. Moreover, any or all parts of the API 612 or the service layer 613 may be implemented as child or sub-modules of another software module, enterprise application, or hardware module without departing from the scope of this disclosure.

Computer system 602 includes an interface 604. Although illustrated as a single interface 604 in FIG. 6, two or more interfaces 604 may be used according to particular needs, desires, or particular implementations of the computer system 602. The interface 604 is used by the computer system 602 for communicating with other systems in a distributed environment that are connected to the network 630. Generally, the interface 604 includes logic encoded in software or hardware (or a combination of software and hardware) and operable to communicate with the network 630. More specifically, the interface 604 may include software supporting one or more communication protocols associated with communications such that the network 630 or interface's hardware is operable to communicate physical signals within and outside of the illustrated computer system 602.

The computer system 602 includes at least one computer processor 605. Although illustrated as a single computer processor 605 in FIG. 6, two or more processors may be used according to particular needs, desires, or particular implementations of computer system 602. Generally, the computer processor 605 executes instructions and manipulates data to perform the operations of computer system 602 and any machine learning networks, algorithms, methods, functions, processes, flows, and procedures as described in the instant disclosure.

Computer system 602 also includes a memory 606 that holds data for computer system 602 or other components (or a combination of both) that can be connected to the network 630. For example, memory 606 can be a database storing data consistent with this disclosure. Although illustrated as a single memory 606 in FIG. 6, two or more memories may be used according to particular needs, desires, or particular implementations of computer system 602 and the described functionality. While memory 606 is illustrated as an integral component of computer system 602, in alternative implementations, memory 606 can be external to computer system 602.

The application 607 is an algorithmic software engine providing functionality according to particular needs, desires, or particular implementations of computer system 602, particularly with respect to functionality described in this disclosure. For example, application 607 can serve as one or more components, modules, applications, etc. Further, although illustrated as a single application 607, the application 607 may be implemented as multiple applications 607 on computer system 602. In addition, although illustrated as integral to computer system 602, in alternative implementations, the application 607 can be external to computer system 602.

There may be any number of computer systems 602 associated with, or external to, a computer system containing a computer system 602, where each computer system 602 communicates over network 630. Further, the term “client,” “user,” and other appropriate terminology may be used interchangeably as appropriate without departing from the scope of this disclosure. Moreover, this disclosure contemplates that many users may use one computer system 602, or that one user may use multiple computer systems 602.

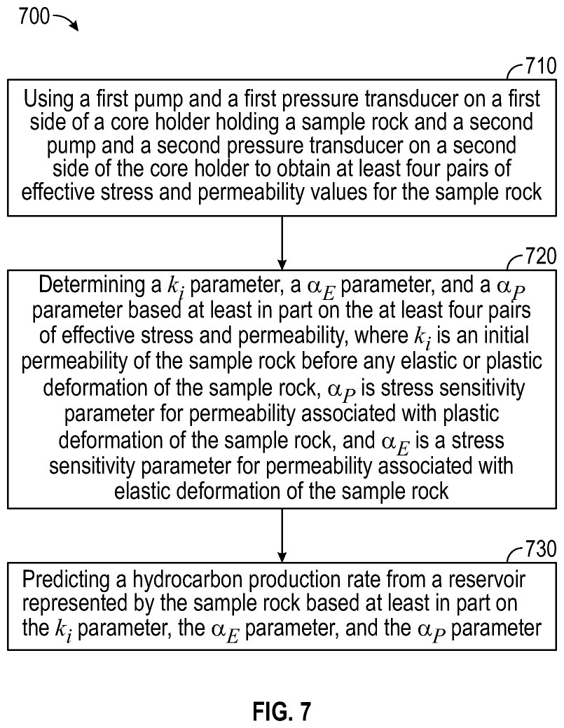

Turning to FIG. 7, a flow diagram 700 shows a method in accordance with some embodiments for predicting hydrocarbon production from an unconventional reservoir. Following flow diagram 700, at least four pairs of effective stress and permeability values for the sample rock are obtained using a first pump and a first pressure transducer on a first side of a core holder holding a sample rock and a second pump and a second pressure transducer on a second side of the core holder (block 710). A kᵢ parameter, an α_{E} parameter, and an α_{P} parameter are determined based at least in part on the at least four pairs of effective stress and permeability (block 720). The kᵢ is an initial permeability of the sample rock before any elastic or plastic deformation of the sample rock, the α_{P} is stress sensitivity parameter for permeability associated with plastic deformation of the sample rock, and the α_{E} is a stress sensitivity parameter for permeability associated with elastic deformation of the sample rock. A hydrocarbon production rate from a reservoir represented by the sample rock is predicted based at least in part on the kᵢ parameter, the α_{E} parameter, and the α_{P} parameter (block 730).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112(f) for any limitations of any of the claims herein, except for those in which the claim expressly uses the words ‘means for’ together with an associated function.

What is claimed is:

1. A method comprising:
using a first pump and a first pressure transducer on a first side of a core holder holding a sample rock and a second pump and a second pressure transducer on a second side of the core holder to obtain at least four pairs of effective stress and permeability values for the sample rock, wherein the sample rock comprises fractures;
determining a kᵢ parameter, an α_{E} parameter, and an α_{P} parameter based at least in part on the at least four pairs of effective stress and permeability; wherein the kᵢ parameter is an initial permeability of the sample rock before any elastic or plastic deformation of the sample rock, the α_{P} parameter is a stress sensitivity parameter for permeability associated with plastic deformation of the sample rock, and the α_{E} parameter is a stress sensitivity parameter for permeability associated with elastic deformation of the sample rock; and
predicting a hydrocarbon production rate from reservoir represented by the sample rock based at least in part on the kᵢ parameter, the α_{E} parameter, and the α_{P} parameter.

2. The method according to claim 1, wherein:
a value for kᵢ and a value for (αₚ+α_{E}) are determined by fitting pairs of effective stress and observed permeability obtained when the sample rock is subject to both elastic and plastic deformation with ln(k)=ln(kᵢ)−(αₚ+α_{E})*σₑ, wherein k is a permeability of the sample rock, and σₑ is an effective stress on the sample rock; and
a value for the α_{E} parameter is determined by fitting pairs of effective stress and observed permeability obtained when the sample rock is subject to elastic deformation with ln(k)=Constant−α_{E}*σₑ.

3. The method according to claim 2, wherein the at least four pairs of effective stress and permeability values for the sample rock are measured by a steady-state flow system comprising: the core holder, the first pump, the second pump, the first pressure transducer, the second pressure transducer, and a third pump; wherein the third pump is a confinement pump configured to generate the effective stress on the sample rock; wherein the first pump is an upstream pump; wherein the second pump is a downstream pump; and wherein a combination of the first pump and the second pump are configured to apply a flow pressure on the sample rock.

4. The method according to claim 3, wherein a permeability of the sample rock is obtained for at least four effective stress values extending across a range from at least five hundred pounds per square inch to at least one thousand pounds per square inch.

5. The method according to claim 2, wherein the at least four pairs of effective stress and permeability values for the sample rock are measured by a pressure pulse decay system comprising: the core holder, the first pump, an upstream reservoir between the first pump and the core holder, the second pump, a downstream reservoir between the second pump and the core holder, the first pressure transducer, the second pressure transducer, and a third pump; wherein the third pump is a confinement pump configured to generate the effective stress on the sample rock; wherein the first pump is an upstream pump; wherein the second pump is a downstream pump; and wherein a combination of the first pump and the second pump are configured to apply a flow pressure on the sample rock.

6. The method according to claim 5, wherein a permeability of the sample rock is obtained for at least four effective stress values extending across a range from at least five hundred pounds per square inch to at least one thousand pounds per square inch.

7. The method according to claim 2, wherein obtaining the at least four pairs of effective stress and permeability comprise loading the sample rock with an effective stress, wherein loading the sample rock with an effective stress comprises:
applying pressure by a third pump to the core holder to yield a first effective stress corresponding to a stress point A, wherein the permeability of the sample rock is zero at the stress point A;
increasing the pressure applied by the third pump to yield a second effective stress corresponding to a stress point B, wherein upon increasing the pressure applied by the third pump, the sample rock begins to deform plastically;
further increasing the pressure applied by the third pump to yield a third effective stress corresponding to a stress point C;
decreasing the pressure applied by the third pump to yield a fourth effective stress corresponding to a stress point D;
wherein the third effective stress is greater than the second effective stress and the fourth effective stress; and
wherein the at least four pairs of effective stress and permeability include at least two pairs of effective stress and permeability correspond to the range between stress point A and the stress point B, and at least two pairs of effective stress and permeability correspond to the range between stress point B and the stress point C;
wherein the at least two pairs of effective stress and permeability correspond to the range between stress point A and the stress point B are used to determine the value for kᵢ and the value for (αₚ+α_{E}); and
wherein the at least two pairs of effective stress and permeability correspond to the range between stress point B and the stress point C are used to determine α_{E}.

8. The method according to claim 7, wherein the loading the sample rock with the effective stress further comprises:
increasing the pressure applied by the third pump to yield the third effective stress corresponding to the stress point C;
further increasing the pressure applied by the third pump to yield a fifth effective stress corresponding to a stress point E;
decreasing the pressure applied by the third pump to yield a sixth effective stress corresponding to a stress point F;
wherein the fifth effective stress is greater than the third effective stress, the fourth effective stress, and the sixth effective stress; and
wherein the at least four pairs of effective stress and permeability include at least two pairs of effective stress and permeability correspond to the range between stress point E and the stress point F, and at least two pairs of effective stress and permeability correspond to the range between stress point C and the stress point E;
wherein the at least two pairs of effective stress and permeability correspond to the range between stress point E and the stress point F are used to determine the value for the kᵢ parameter and the value for (αₚ+α_{E}); and
wherein the at least two pairs of effective stress and permeability correspond to the range between stress point C and the stress point E are used to determine the α_{E} parameter.

9. The method according to claim 8, wherein the sixth effective stress is greater than the fourth effective stress.

10. A system, the system comprising:
a core holder configured to hold a sample rock, wherein the sample rock comprises fractures;
a first pump;
a first pressure transducer, wherein the first pump and the first pressure transducer are located on an inlet side of the core holder;
a second pump;
a second pressure transducer, wherein the second pump and the second pressure transducer are located on an outlet side of the core holder;
a third pump;
a third pressure transducer, wherein the third pump is configured to control an effective stress on the sample rock, and wherein the third pressure transducer is configured to sense the pressure generated by the third pump on the sample rock;
a reservoir characterization device configured to:
receive first pressure measurements from the first pressure transducer, second pressure measurements from the second pressure transducer, and third pressure measurements from the third pressure transducer;
generate at least four pairs of effective stress and permeability based upon a combination of the first pressure measurements, the second pressure measurements, and the third pressure measurements;
determine a kᵢ parameter, a α_{E} parameter, and a α_{P} parameter based at least in part on the at least four pairs of effective stress and permeability; wherein kᵢ is an initial permeability of the sample rock before any elastic or plastic deformation of the sample rock, α_{P} is stress sensitivity parameter for permeability associated with plastic deformation of the sample rock, and α_{E} is a stress sensitivity parameter for permeability associated with elastic deformation of the sample rock; and
predict a hydrocarbon production rate from reservoir represented by the sample rock based at least in part on the kᵢ parameter, the α_{E} parameter, and the α_{P} parameter.

11. A computer readable medium, the computer readable medium having stored therein instructions which, when executed by one or more processors perform the following method:
receiving first pressure measurements from a first pressure transducer measuring pressure at an inlet side of a core holder holding a sample rock;
receiving second pressure measurements from a second pressure transducer measuring pressure at an outlet side of the core holder;
receiving third pressure measurements from a third pressure transducer measuring an effective stress on the sample rock;
generating at least four pairs of effective stress and permeability based upon a combination of the first pressure measurements, the second pressure measurements, and the third pressures measurements;
determining a kᵢ parameter, a α_{E} parameter, and a α_{P} parameter based at least in part on the at least four pairs of effective stress and permeability; wherein the kᵢ parameter is an initial permeability of the sample rock before any elastic or plastic deformation of the sample rock, the α_{P} parameter is stress sensitivity parameter for permeability associated with plastic deformation of the sample rock, and the α_{E} parameter is a stress sensitivity parameter for permeability associated with elastic deformation of the sample rock; and
predicting a hydrocarbon production rate from reservoir represented by the sample rock based at least in part on the kᵢ parameter, the α_{E} parameter, and the α_{P} parameter.

12. The computer readable medium of claim 11, wherein:
a value for the kᵢ parameter and a value for (α_{P}+α_{E}) are determined by fitting pairs of effective stress and observed permeability obtained when the sample rock is subject to both elastic and plastic deformation with ln(k)=ln(kᵢ)−(α_{P}+α_{E})*σₑ, wherein k is a permeability of the sample rock, and σₑ is an effective stress on the sample rock; and
a value for the α_{E} parameter is determined by fitting pairs of effective stress and observed permeability obtained when the sample rock is subject to elastic deformation with ln(k)=Constant−α_{E}*σₑ.

13. The computer readable medium of claim 12, wherein the at least four pairs of effective stress and permeability values for the sample rock are measured by a steady-state flow system comprising: the core holder, the first pump, the second pump, the first pressure transducer, the second pressure transducer, the third pressure transducer, and the third pump; wherein the third pump is a confinement pump; wherein the first pump is an upstream pump; wherein the second pump is a downstream pump; and wherein a combination of the first pump and the second pump are configured to apply a flow pressure on the sample rock.

14. The computer readable medium of claim 13, wherein a permeability of the sample rock is generated for at least four effective stress values extending across a range from at least five hundred pounds per square inch to at least one thousand pounds per square inch.

15. The computer readable medium of claim 13, wherein the at least four pairs of effective stress and permeability values for the sample rock are measured by a pressure pulse decay system comprising: the core holder, the first pump, an upstream reservoir between the first pump and the core holder, the second pump, a downstream reservoir between the second pump and the core holder, the first pressure transducer, the second pressure transducer, the third pressure transducer, and the third pump; wherein the third pump is a confinement pump; wherein the first pump is an upstream pump; wherein the second pump is a downstream pump; and wherein a combination of the first pump and the second pump are configured to apply a flow pressure on the sample rock.

16. The computer readable medium of claim 15, wherein a permeability of the sample rock is generated for at least four effective stress values extending across a range from at least five hundred pounds per square inch to at least one thousand pounds per square inch.

17. The computer readable medium of claim 12, wherein generating the at least four pairs of effective stress and permeability comprise loading the sample rock with an effective stress, wherein loading the sample rock with the effective stress comprises:
applying pressure by the third pump to the core holder to yield a first effective stress corresponding to a stress point A, wherein the permeability of the sample rock is zero at the stress point A;
increasing the pressure applied by the third pump to yield a second effective stress corresponding to a stress point B, wherein upon increasing the pressure applied by the third pump, the sample rock begins to deform plastically;
further increasing the pressure applied by the third pump to yield a third effective stress corresponding to a stress point C;
decreasing the pressure applied by the third pump to yield a fourth effective stress corresponding to a stress point D;
wherein the third effective stress is greater than the second effective stress and the fourth effective stress; and
wherein the at least four pairs of effective stress and permeability include at least two pairs of effective stress and permeability correspond to the range between stress point A and the stress point B, and at least two pairs of effective stress and permeability correspond to the range between stress point B and the stress point C;
wherein the at least two pairs of effective stress and permeability correspond to the range between stress point A and the stress point B are used to determine the value for the kᵢ parameter and the value for (α_{P}+α_{E}); and
wherein the at least two pairs of effective stress and permeability correspond to the range between stress point B and the stress point C are used to determine the α_{E} parameter.

18. The computer readable medium of claim 17, wherein the loading the sample rock with the effective stress further comprises:
increasing the pressure applied by the third pump to yield the third effective stress corresponding to the stress point C;
further increasing the pressure applied by the third pump to yield a fifth effective stress corresponding to a stress point E;
decreasing the pressure applied by the third pump to yield a sixth effective stress corresponding to a stress point F;
wherein the fifth effective stress is greater than the third effective stress, the fourth effective stress, and the sixth effective stress; and
wherein the at least four pairs of effective stress and permeability include at least two pairs of effective stress and permeability correspond to the range between stress point E and the stress point F, and at least two pairs of effective stress and permeability correspond to the range between stress point C and the stress point E;
wherein the at least two pairs of effective stress and permeability correspond to the range between stress point E and the stress point F are used to determine the value for the kᵢ parameter and the value for (α_{P}+α_{E}); and
wherein the at least two pairs of effective stress and permeability correspond to the range between stress point C and the stress point E are used to determine the α_{E} parameter.

19. The computer readable medium of claim 18, wherein the sixth effective stress is greater than the fourth effective stress.
